(19) United States
(12) Patent Application Publication
(10) Pub. No.: US 2006/0025468 A1
(43) Pub. Date: Feb. 2, 2006

(54) Chiral pyrrolidine derivatives, and methods for preparing compounds thereof

(76) Inventors: Jeffrey P. Whitten, Santee, CA (US); Michael Schwaebe, San Diego, CA (US); Collin Regan, Los Angeles, CA (US)

Correspondence Address: MORRISON & FOERSTER LLP, 12531 HIGH BLUFF DRIVE, SUITE 100, SAN DIEGO, CA, 92130-2040, US

(21) Appl. No.: 10/903,810

(22) Filed: Jul. 29, 2004

(51) Int. Cl. A61K 31/4025; C07D 403/02; C07D 405/02; C07D 409/02

(52) U.S. Cl. 514/422; 548/517

(57) ABSTRACT

The present invention relates to chiral pyrrolidine derivatives; and methods for preparing compounds thereof. The chiral pyrrolidine derivatives of the present invention may be used as intermediates for pharmaceutical and agricultural chemicals.

### TECHNICAL FIELD

The invention relates to the field of organic chemistry, particularly synthetic methodologies for preparing chiral compounds.

### BACKGROUND ART

Pyrrolidines are ubiquitous structural motifs in drugs and drug candidates exhibiting antidepressant, antihypertensive, anti-arthritic, antibacterial, antithrombotic and analgesic activities. (See, e.g., WO 89/06534, JP-1996092207; WO 95/08534, U.S. Pat. No. 5,665,754, Bioorg. Med. Chem. Lett. (1998) 8:2833-2838, U.S. Pat. No. 5,668,147, J. Pharm. Pharmacol. (1996) 48:380-385, and U.S. Pat. No. 5,514,701, each incorporated herein by reference.) Furthermore, recent drug development incorporating the pyrrolidine motif has identified candidates with promising anti-HIV and antibacterial adjunct activities. (See, e.g., Bioorg. Med. Chem. Lett. (2001) 11:2741-2745, U.S. Pat. No. 6,166,037, WO 00/01714 and U.S. Pat. No. 6,399,629.) Although various pyrrolidine intermediates with a variety of functional groups are commercially available, most compounds are only available as racemates.

Many commercially marketed drugs have at least one chiral center (e.g., ibuprofen), and sold as racemic mixtures. Often, one enantiomer has the desired therapeutic activity whereas the other enantiomer causes undesirable side-effects and may limit drug efficacy or dosage. Thus, the development of economical methods for preparing chiral intermediates such as pyrrolidine intermediates for pharmaceutical and agricultural chemicals is highly desirable.

### DISCLOSURE OF THE INVENTION

The present invention relates to chiral pyrrolidine derivatives, and methods for preparing such compounds.

In one aspect, the present invention relates to a method for preparing a compound having formula (1) or (2) 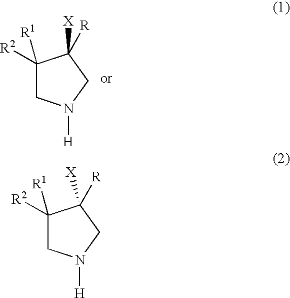
comprising:
a) separating from a racemate, chiral isomers having the formula (3) 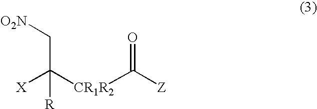
b) reducing the nitro group to an amino group to form a lactam; and
c) reducing the carbonyl group in the lactam to a methylene group,
wherein each R, R¹ and R² are independently H, optionally substituted alkyl optionally containing one or more N, O or S; aryl, heteroaryl or heterocyclic ring, provided R¹ and R² are identical;
each X is a 5-14 membered ring, optionally substituted with halo, optionally substituted alkyl optionally containing one or more N, O or S; aryl, heteroaryl or heterocyclic ring; and
Z is a removable chiral group.

In one example, formula (3) in step a) is formed by contacting a compound having the formula X—CR═CH—NO₂ wherein R is as defined above, with an enolate having a chiral leaving group. In a particular example, the enolate may comprise acetyl enolate.

In another example, step c) comprises converting the lactam obtained from step b) to a thiolactam, and converting the thiolactam to a methylene group.

In yet another example, step a) comprises separating chiral isomers via chromatography. The methods may further comprise recrystallizing the chiral isomers.

In one example, the chiral isomers are diastereomers. In particular examples, the diastereomers have different polarities.

In the above formula (1) or (2), X may be phenyl, furan, tetrahydrofuran, 1,3-dioxolane, 2,3-dihydrofuran, tetrahydropyran, benzofuran, isobenzofuran, 1,3-dihydro-isobenzofuran, oxazole, isoxazole, 4,5-dihydroisoxazole, piperidine, pyrrolidine, pyrrolidin-2-one, pyrrole, pyridine, pyrimidine, octahydro-pyrrolo[3,4-b]pyridine, piperazine, pyrazine, morpholine, thiomorpholine, imidazole, imidazolidine-2,4-dione, benzimidazole, 1,3-dihydrobenzimidazol-2-one, indole, thiazole, benzothiazole, thiadiazole, thiophene, tetrahydro-thiophene 1,1-dioxide, diazepine, triazole, triazine, diazabicyclo[2.2.1]heptane, 2,5-diazabicyclo[2.2.1]heptane, or 2,3,4,4a,9,9a-hexahydro-1H-β-carboline, each of which is optionally substituted.

In particular embodiments, X is pyrazine, pyrimidine, thiophene, furan, imidazole, thiazole, triazine, or oxazole. In one particular example, X is pyrazine.

In the above formula (1) or (2), each R may be H.

In the above formula (1) or (2), Z may comprise an oxazolidinonyl group. For example, Z may be N-acetyl-(4R)-benzyl-2-oxazolidinone and derivatives thereof.

The present invention also provides compounds having formula (1) or (2), as defined above. In one example, the compound is (R)-3-(S)-4-nitro-3-(pyrazin-2-yl)butanoyl)-4-benzyloxazolidin-2-one or (R)-3-((R)-4-nitro-3-(pyrazinyl-2-yl)butanoyl)-4-benzyloxazolidin-2-one.

Furthermore, the present invention provides a method for preparing compounds of formula (4) or (5) 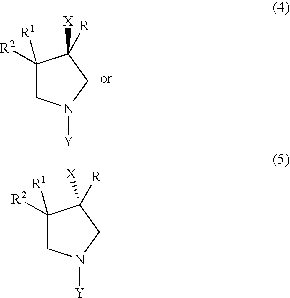
comprising contacting a compound having formula (1) or (2) as defined above, with a compound Y-L, wherein Y is the residue of a compound having a therapeutic or agricultural effect and L is a leaving group, to form a chiral N-substituted pyrrolidinyl compound having formula (4) or (5).

In the above method, the leaving group L may be halo, tosylate, carbonate, acetate, carbamate, trifluoroacetate, or phosphate.

The above method may further comprise determining the therapeutic or agricultural effect of the N-substituted pyrrolidinyl compound having formula (4) or (5) in a target system.

The target system may be a cell or tissue. In one example, the target system is associated with cancer.

The present invention also relates to compounds having formula (4) or (5), as well as pharmaceutical and agricultural compositions comprising such compounds. In one aspect, the present invention provides a therapeutically effective N-substituted pyrrolidinyl compound having formula (4) or (5), and pharmaceutical compositions thereof. In one example, compounds having formula (4) or (5) may comprise a quinobenzoxamine or 1,4-dihydro-4-oxo-napthypyridinyl ring. In particular examples, compounds having formula (4) or (5) may exhibit antidepressant, antiarthritic, antimicrobial, antibacterial, antihypertensive, antiviral, antithrombotic or antiproliferative properties. In other example, the

In another aspect, the present invention provides an agriculturally effective N-substituted pyrrolidinyl compound having formula (4) or (5). In particular examples, compounds having formula (4) or (5) may be suitable for application to crops, soil or animals.

Definitions

The singular forms “a”, “an”, and “the” include plural references, unless explicitly indicated otherwise.

As used herein, a chiral compound is a compound that is different from its mirror image, and has an enantiomer. Methods of synthesizing chiral compounds and resolving a racemic mixture of enantiomers are well known to those skilled in the art. See, e.g., March, “Advanced Organic Chemistry,” John Wiley and Sons, Inc., New York (1985), which is incorporated herein by reference.

As used herein, the term “alkyl” refers to a carbon-containing compound, and encompasses compounds containing one or more heteroatoms, and compounds substituted with one or more substituents known in the art.

As used herein, the term “heterocycle” refers to a cyclic compound comprising a heteroatom, including monocyclic or fused heterocycles. The term “heteroatom” refers to any atom that is not carbon or hydrogen, such as nitrogen, oxygen or sulfur. Examples of heterocycles include but are not limited to oxirane, oxetane, pyran, tetrahydropyran, dioxane, lactones, aziridine, azetidine, pyrrolidine, piperidine, morpholine, lactams, and tetrahydrofuran. Examples of fused heterocycles include but are not limited to benzimidazole, benzofuran, benzoxazole, isobenzofuran, and the like.

As used herein, the terms “heteroaryl” or “heteroaromatic” refer to an aromatic heterocycle. Examples of heteroaryls include but are not limited to furan, pyrrole, pyridine, pyrimidine, imidazole, triazole, and the like.

As used herein, the term “linker” refers to intervening atoms between the substituent “X” and the pyrrolidinyl moiety.

The terms “therapeutic effect” or “therapeutically effective” as used herein refer to the pharmacological activity of a compound, as known to the skill in the art. Examples of a compound's pharmacological properties include but are not limited to antidepressant, antiarthritic, antimicrobial, antibacterial, antihypertensive, antiviral, antithrombotic, and antiproliferative properties.

As used herein, the term “antiproliferative properties” refers to reducing or stopping a cell proliferation rate (e.g., slowing or halting tumor growth) or reducing the number of proliferating cancer cells (e.g., removing part or all of a tumor). These terms also are applicable to reducing a titre of a microorganism in a system (i.e., cell, tissue, or subject) infected with a microorganism, reducing the rate of microbial propagation, reducing the number of symptoms or an effect of a symptom associated with the microbial infection, and/or removing detectable amounts of the microbe from the system. Examples of microorganism include but are not limited to virus, bacterium and fungus.

As used herein, the terms “agricultural effect” or “agriculturally effective” refer to the agricultural activity of a compound, as known to the skill in the art. Examples of a compound's agricultural activity include but are not limited to its use as a pesticide, defoliant, desiccant or a plant nutrient. Examples of agricultural compounds and compositions are described in U.S. Pat. Nos. 5,877,112; 5,278,132; 5,145,954; 4,492,598; and 4,440,565, which are incorporated herein by reference.

### MODES OF CARRYING OUT THE INVENTION

The present invention relates to chiral pyrrolidine derivatives, and methods for preparing such compounds. In particular, In one aspect, the present invention relates to a method for preparing a compound having formula (1) or (2) 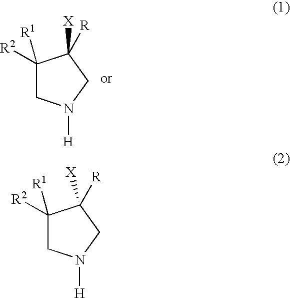
comprising:
a) separating from a racemate, chiral isomers having the formula (3) 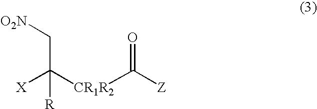
b) reducing the nitro group to an amino group to form a lactam; and
c) reducing the carbonyl group in the lactam to a methylene group,
wherein each R, R¹ and R² are independently H, optionally substituted alkyl optionally containing one or more N, O or S; aryl, heteroaryl or heterocyclic ring, provided R¹ and R² are identical;
each X is a 5-14 membered ring, optionally substituted with halo, optionally substituted alkyl optionally containing one or more N, O or S; aryl, heteroaryl or heterocyclic ring; and
Z is a removable chiral group.

In the above formula (1) or (2), the linker may be a covalent bond or a chemical functional group that directly connects the pyrrolidinyl moiety to substituent “X”. The linker may comprise a series of covalently bonded atoms and their substituents, and may collectively be referred as a linking group. In preferred embodiments, the linker is a covalent bond. Alternatively, the linker may be chosen to serve as a spacer between the pyrrolidinyl moiety and substituent “X”, or to relieve steric hindrance that may interfere with compound activity and/or pharmacological effect. A variety of linking moieties are known to those of skill in the art, which may be used compounds of the present invention.

In the above formula (1) or (2), X is an optionally substituted 5-14 membered ring. Examples of 5-14 membered rings include but are not limited to phenyl, tetrahydrofuran, 1,3-dioxolane, 2,3-dihydrofuran, tetrahydropyran, benzofuran, isobenzofuran, 1,3-dihydro-isobenzofuran, isoxazole, 4,5-dihydroisoxazole, piperidine, pyrrolidine, pyrrolidin-2-one, pyrrole, pyridine, pyrimidine, octahydro-pyrrolo[3,4-b]pyridine, piperazine, pyrazine, morpholine, thiomorpholine, imidazole, imidazolidine-2,4-dione, benzimidazole, 1,3-dihydrobenzimidazol-2-one, indole, thiazole, benzothiazole, thiadiazole, thiophene, tetrahydro-thiophene 1,1-dioxide, diazepine, triazole, guanidine, diazabicyclo[2.2.1]heptane, 2,5-diazabicyclo[2.2.1]heptane, and 2,3,4,4a,9,9a-hexahydro-1H-β-carboline.

In particular examples, X is an optionally substituted pyrazine, pyrimidine, pyridine, thiophene, furan, imidazole, thiazole, triazine or oxazole. In one example, X is pyrazine. The methods of the present invention are also contemplated for preparing chiral 2- and 3-substituted pyrrolidine intermediates shown in Tables 1-2.

TABLE 1
2-substituted Pyrrolidines

2-phenylpyrrolidine
2-(4-methoxyphenyl)pyrrolidine
2-(4-chlorophenyl)pyrrolidine
2-(3,5-dichlorophenyl)pyrrolidine
2-(4-fluorophenyl)pyrrolidine
2-[4-(trifluoromethyl)phenyl]pyrrolidine
2-(2-chlorophenyl)pyrrolidine
2-(2-methylphenyl)pyrrolidine
2-[2-(trifluoromethyl)phenyl]pyrrolidine hydrochloride
2-benzylpyrrolidine
2-(2-phenylethyl)pyrrolidine
2-pyrrolidin-2-ylpyridine
3-pyrrolidin-2-ylpyridine
4-pyrrolidin-2-ylpyridine
3-pyrrolidin-2-ylmethylpyridine dihydrochloride
2-cyclohexylpyrrolidine
2-(methylethyl)pyrrolidine hydrochloride
2-(2-methylpropyl)pyrrolidine hydrochloride

TABLE 2
3-substituted Pyrrolidines

3-phenylpyrrolidine
3-benzylpyrrolidine
3-(pyrrolidin-3-ylmethyl)pyridine dihydrochloride
3-(4-methoxybenzyl)pyrrolidine oxalate
3-(4-chlorobenzyl)pyrrolidine oxalate
3-(4-fluorobenzyl)pyrrolidine hydrochloride
3-(4-methylbenzyl)pyrrolidine hydrochloride
3-(4-trifluoromethylbenzyl)pyrrolidine oxalate
3-(2-methoxybenzyl)pyrrolidine oxalate
3-(2-chlorobenzyl)pyrrolidine oxalate
3-(2-phenylethyl)pyrrolidine
2-(pyrrolidin-3-yl)pyridine
3-(pyrrolidin-3-yl)pyridine
4-(pyrrolidin-3-yl)pyridine hydrochloride
2-(pydrrolidin-3-yl)pyrazine trihydrochloride
2-(pyrrolidin-3-yl)pyrimidine trihydrochloride
3-(trifluoromethyl)pyrrolidine
3-(benzenesulfonyl)pyrrolidine
3-(methanesulfonyl)pyrrolidine

In the above formula (1) or (2), each X and R each may optionally be substituted with one or more substituents. The term “substituted” as used herein refers to a replacement of an atom or chemical group with a functional group. Examples of substituents include but are not limited to NR₂, —OR, —SR, carbonyl compounds such as aldehydes, ketones and esters, heterocycle, aryl, alkyl, alkenyl, alkynyl, ionic groups (e.g., —NR₃⁺), halo, inorganic substituents, amide and amide derivatives, and other substituents known to those skilled in the art. The term “substituted” also includes multiple degrees of substitution.

In the above formula (1) or (2), Z is any suitable removable chiral group or chiral auxiliary for transferring chirality known in the art. In a particular example, Z is an oxazolidinonyl leaving group. Other contemplated removable chiral groups include chiral oxazolidinethiones and thiazolidinethiones, such as those described in Tables 3 and 4. (See, e.g., Sakane, et al., J. Am. Chem. Soc. (1983) 105:6165-6155; Velazquez, et al., Curr. Org. Chem. (2002) 6:1-38).

TABLE 3
1,3-Oxazolidine-2-thiones

| | | |
|---|---|---|
| 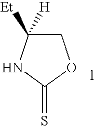 | 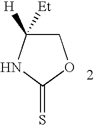 | 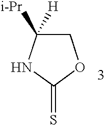 |
| 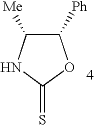 | 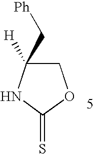 | 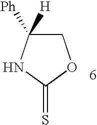 |
| 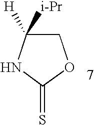 | 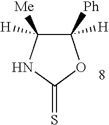 | 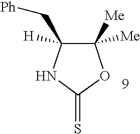 |
| 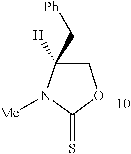 | 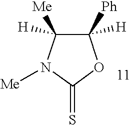 | |

TABLE 4
Thiazolidinethiones

| | | |
|---|---|---|
| 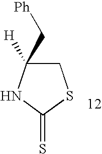 | 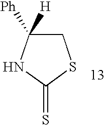 | 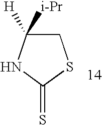 |
| 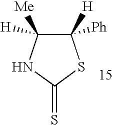 | 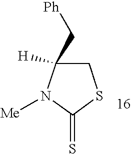 | 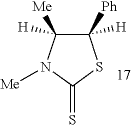 |

Acylation of oxazolidinones, oxazolidinethiones and thiazolidinethiones may be accomplished, for example, by treatment of these compounds with an alkyl lithium and an appropriate acyl chloride. Alternatively, the oxazolidinones, oxazolidinethiones or thiazolidinethiones may be treated with the corresponding acyl chlorides in the presence of triethylamine. Other methodologies for N-acylation are also known, such as the condensation of acyl chlorides with thalium (I) salts of thiazolidinethiones, and condensations of carboxylic acids with thiazolidinethiones in the presence of dicyclohexylcarbodiimide.

In another aspect, the present invention relates to a method for preparing compounds (1) or (2), wherein R, n and X are as defined above, comprising: a) contacting a compound having the formula X—CR═CR—NO₂, wherein R is as defined above, with a chiral enolate having a leaving group to form a racemate; b) separating chiral isomers from the racemate; c) reducing the nitro group from the chiral isomers to an amino group to form a lactam; and d) reducing the carbonyl group in the lactam to a methylene group. In particular embodiments, the chiral isomers have formula (3), as defined above.

In the above method, step d) may further comprise converting the lactam obtained from step c) to a thiolactam, and converting the thiolactam to a methylene group. Step b) may further comprise separating chiral isomers from the racemate via chromatography, and optionally recrystallizing the chiral isomers.

One method for preparing chiral pyrrolidinyl derivatives is shown in Scheme 1.

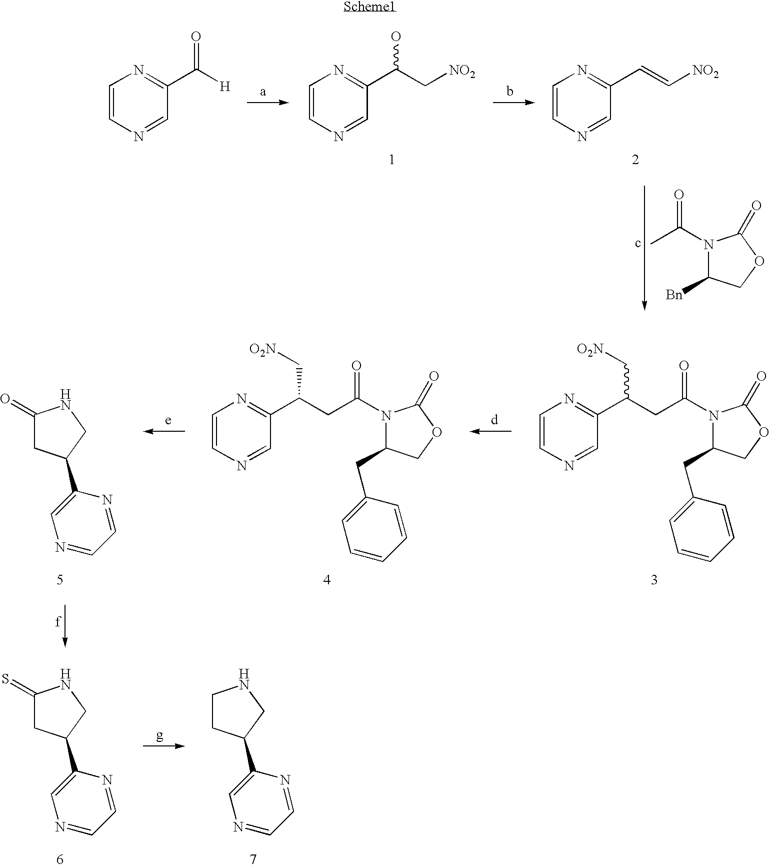

a) 1.5 eq CH₃NO₂, 0.1 eq KOt-bu, THF, 0° C., 64%; b) i) 1.1 eq TFAA, CH₂Cl₂, −10° C.; ii) 2.1 eq TEA, CH₂Cl₂, −10° C., 82%; c) 1.1 eq Na(NSiMe₃)₂, THF, −78° C., 65%; d) separate diastereomers; e) Raney Ni, H₂ (1 atm), EtOH, 68%; f) Lawesson's Reagent, toluene, 80° C., 75%; g) Raney Ni, EtOH, H₂ (1 atm), 50° C., 35%.

As shown in scheme 1, nitromethane (11.1 mL, 207 mmol, 1.5 eq) was added to a solution of the pyrazine aldehyde (15 g, 138 mmol) in 35 mL of anhydrous THF. The solution was cooled to 0° C. Potassium tert-butoxide (13.8 mL of 1 M solution in THF, 13.8 mmol, 0.1 eq) was added dropwise keeping the solution temperature below 5° C. A yellow precipitate formed upon addition of base. After addition of base was complete, the reaction was stirred for 15 minutes at 0° C. The solution was poured into 250 mL of water and extracted with EtOAc (3×250 mL). The combined organics were washed with brine, dried over Na₂SO₄, filtered, and concentrated in vacuo. The residue was purified via silica plug and eluted with 60% EtOAc/hexanes. The product (1) was a yellow viscous oil (15.1 g, 64%). LC/MS (M+1) 170.

A solution of (1) (15.1 g, 89.3 mmol) in 110 mL of anhydrous CH₂Cl₂ was cooled to −20° C. Trifluoroacetic anhydride (13.6 mL, 98.2 mmol, 1.1 eq) was added with the solution being kept below −10° C. The solution was stirred for two minutes after addition of the TFAA was complete. Then triethylamine (26 mL, 187.5 mmol, 2.1 eq) was added dropwise with the solution being kept below −10° C. The solution became homogeneous and orange/brown upon addition of base. The solution was stirred for 15 minutes after addition of the TEA was complete. The solution was diluted with 200 mL of CH₂Cl₂ and washed with a saturated ammonium chloride solution. The aqueous layer was extracted with CH₂Cl₂ (2×250 mL). The combined organics were washed with brine, dried over Na₂SO₄, filtered, and concentrated in vacuo. The crude brown solid residue was suspended in 300 mL of hot EtOAc and filtered. The filtrate was concentrated in vacuo. The residue was purified via silica plug and eluted with 50% EtOAc/hexanes. The nitrostyrene intermediate (2) was a bright yellow solid (11.13 g, 82%). GC/MS (151).

N-Acetyl-(4R)-benzyl-2-oxazolidinone (7.54 g, 34.4 mmol) was dissolved in 200 mL anhydrous THF and the solution was cooled to −78° C. Sodium hexamethyldisilylazide (34.4 mL of 1 M THF solution, 34.4 mmol, 1.0 eq) was added dropwise with the solution being kept below −65° C. The enolate was formed over one hour. The nitrostyrene intermediate (2) (5.2 g, 34.4 mmol, 1.0 eq) was dissolved in 30 mL of anhydrous THF and added dropwise via syringe keeping the solution temperature below −65° C. The reaction was stirred for 2 hours at −78° C. and then quenched with 50 mL of saturated ammonium chloride solution at −78° C. The solution was partitioned between EtOAc and H₂O (500 mL each). The aqueous layer was extracted with EtOAc (1×500 mL). The combined organics were washed with brine, dried over Na₂SO₄, filtered, and concentrated in vacuo. The crude solid was purified via flash column eluting with 60% EtOAc/hexanes to yield approximately 50/50 mixture of diastereomers (3) in 65% yield. LC/MS (M+1) 371.

The pure diastereomers (4) were obtained by first enriching the separate diastereomers via a flash column with a large volume of silica gel eluting with 60% EtOAc/hexanes. The enriched less polar diastereomer (by TLC) was recrystallized in hot MeOH. The resulting colorless needles were free of the more polar diastereomer by NMR. LC/MS (M+1) 371.

X-ray crystal analysis confirmed that this product was (R)-3-(S)-4-nitro-3-(pyrazin-2-yl)butanoyl)-4-benzyloxazolidin-2-one. The enriched more polar diastereomer, (R)-3-((R)-4-nitro-3-(pyrazinyl-2-yl)butanoyl)-4-benzyloxazolidin-2-one was recrystallized in 2-propanol. The resulting light, clumpy white solid was free of the other less polar diastereomer by NMR. LC/MS (M+1) 371. ¹H NMR (CDCl₃): δ 2.75

The more polar (R,R) diastereomer (1.57 g, 4.2 mmol) was suspended in 20 mL of CH₃OH. Raney Nickel (4.72 g of H₂O slurry) was added to the solution. The solution was saturated with hydrogen gas and kept under positive H₂ pressure. The reaction was heated to 50° C. for 3 hours. The solution was purged with argon and filtered over celite. The filtrate was concentrated in vacuo. The residue was purified via column chromatography eluting with 5% MeOH/CH₂Cl₂. The white solid (5) was produced in 40% yield. GC/MS (163). The less polar (R,S) diastereomer (0.9 g, 2.4 mmol) was treated with Raney nickel (2.7 g of H₂O slurry) following similar conditions, to give a white solid in 60% yield. GC/MS (163).

The lactam (265 mgs, 1.6 mmol) derived from the more polar (R,R) diastereomer of (3) was suspended in 16 mL of anhydrous toluene. Lawesson's Reagent (970 mgs, 2.4 mmol, 1.5 eq) was added and the solution was heated to 80° C. The reaction was complete by TLC in 10 minutes. The solution was diluted with H₂O and the product was extracted with EtOAc (3×50 mL). The combined organics were washed with brine, dried over Na₂SO₄, filtered, and concentrated in vacuo. The crude product was purified via column chromatography eluting with 5% MeOH/CH₂Cl₂, giving a faint maize colored solid (6) in 68% yield. GC/MS (179). The lactam derived from the less polar (R,S) diastereomer of (3) (240 mgs, 1.47 mmol) was treated Lawesson's Reagent (892 mgs, 2.2 mmol, 1.5 eq) following similar conditions, to give an off-white solid in 75% yield. (GC/MS) 179.

The thiolactam (183 mgs, 1.0 mmol) derived from the more polar (R, R) diastereomer of (3) was suspended in 7 mL of ethanol. Raney Nickel (2.5 g of H₂O slurry) was added. The solution was saturated with H₂, kept under positive H₂ pressure, and heated to 50° C. for 24 hours. The reaction was purged with argon and filtered. The filtrate was concentrated in vacuo. The crude product was purified via column chromatography using alumina. The product eluted with 30% MeOH/CH₂Cl₂ to give a brown oil in 35% yield. GC/MS (149). The thiolactam derived from the less polar (R,S) diastereomer of (3) (284 mgs, 1.6 mmol) was treated with Raney nickel following similar conditions.

The chiral pyrrolidine intermediate derivatives of the present invention are useful intermediates for preparing pharmaceutical and agricultural chemicals. For example, pyrrolidine intermediate derivatives of the present invention may be used to prepare substituted quinobenzoxamine analogs described in co-pending U.S. patent application Ser. No. 10/821,243, which is incorporated by reference herein. Pyrrolidine intermediate derivatives of the present invention may also be used to prepare heterocyclic substituted 1,4-dihydro-4-oxo-napthpyridine analogs described in co-pending U.S. patent application Ser. No. 10/820,487, which is incorporated by reference herein.

It is also contemplated that the pyrrolidine intermediate derivatives of the present invention will be used as intermediates for other pharmaceutical and agricultural chemicals known to those skilled in the art. Table 5 illustrates various pharmaceutical and agricultural chemicals which may be prepared using the intermediates of the present invention. (See, e.g., WO 89/06534, WO 95/08534, U.S. Pat. No. 5,665,754, U.S. Pat. No. 5,668,147, WO 00/01714, JP-1996092207, U.S. Pat. No. 6,166,037, U.S. Pat. No. 5,514,701, WO 01/89519).

TABLE 5

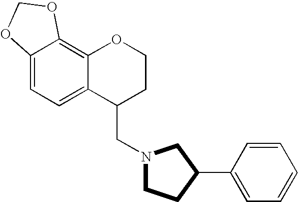

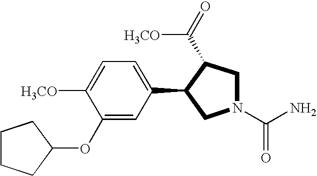

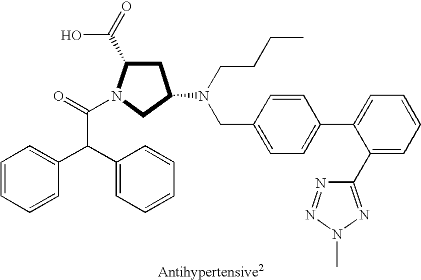

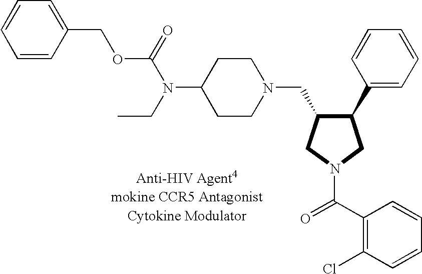

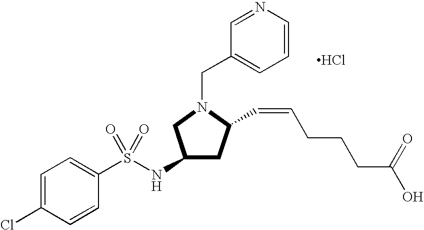

It is understood that the foregoing detailed description and accompanying examples are merely illustrative, and are not to be taken as limitations upon the scope of the invention. Various changes and modifications to the disclosed embodiments will be apparent to those skilled in the art. Such changes and modifications, including without limitation those relating to the chemical structures, substituents, derivatives, intermediates, syntheses, formulations and/or methods of use of the invention, may be made without departing from the spirit and scope thereof. U.S. patents and publications referenced herein are incorporated by reference.

1. A method for preparing a compound having formula (1) or (2) 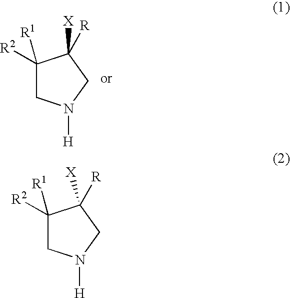
comprising:
a) separating from a racemate, chiral isomers having the formula (3) 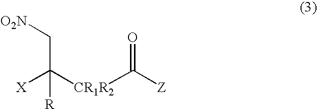
b) reducing the nitro group to an amino group to form a lactam; and
c) reducing the carbonyl group in said lactam to a methylene group,
wherein each R, R¹ and R² are independently H, optionally substituted alkyl optionally containing one or more N, O or S; aryl, heteroaryl or heterocyclic ring, provided R¹ and R² are identical;
each X is a 5-14 membered ring, optionally substituted with halo, optionally substituted alkyl optionally containing one or more N, O or S; aryl, heteroaryl or heterocyclic ring; and
Z is a chiral leaving group.

2. The method of claim 1, wherein formula (3) is formed by contacting a compound having the formula X—CR═CH—NO₂ wherein R is as defined above, with an enolate having a chiral leaving group.

3. The method of claim 1, wherein said chiral isomers are diastereomers.

4. The method of claim 3, wherein said diastereomers have different polarities.

5. The method of claim 1, wherein X is phenyl, furan, tetrahydrofuran, 1,3-dioxolane, 2,3-dihydrofuran, tetrahydropyran, benzofuran, isobenzofuran, 1,3-dihydro-isobenzofuran, oxazole, isoxazole, 4,5-dihydroisoxazole, piperidine, pyrrolidine, pyrrolidin-2-one, pyrrole, pyridine, pyrimidine, octahydro-pyrrolo[3,4-b]pyridine, piperazine, pyrazine, morpholine, thiomorpholine, imidazole, imidazolidine-2,4-dione, benzimidazole, 1,3-dihydrobenzimidazol-2-one, indole, thiazole, benzothiazole, thiadiazole, thiophene, tetrahydro-thiophene 1,1-dioxide, diazepine, triazole, triazine, diazabicyclo[2.2.1]heptane, 2,5-diazabicyclo[2.2.1]heptane, or 2,3,4,4a,9,9a-hexahydro-1H-β-carboline, each of which is optionally substituted.

6. The method of claim 5, wherein X is pyrazine, pyrimidine, thiophene, furan, imidazole, thiazole, triazine, or oxazole.

7. The method of claim 6, wherein X is pyrazine.

8. The method of claim 1, wherein each R is H.

9. The method of claim 1, wherein Z is an oxazolidinonyl group.

10. The method of claim 2, wherein said enolate is acetyl enolate.

11. The method of claim 1, wherein step c) comprises converting said lactam obtained from step b) to a thiolactam, and converting said thiolactam to a methylene group.

12. The method of claim 1, wherein step a) comprises separating chiral isomers via chromatography.

13. The method of claim 12, further comprising recrystallizing said chiral isomers.

14. A compound having formula (1) or (2) 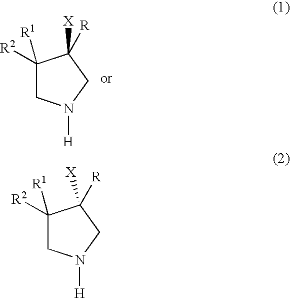
wherein each R, R¹ and R² is independently H, optionally substituted alkyl optionally containing one or more N, O or S; aryl, heteroaryl or heterocyclic ring, provided R¹ and R² are identical; and
each X is a 5-14 membered ring, optionally substituted with halo, optionally substituted alkyl optionally containing one or more N, O or S; aryl, heteroaryl or heterocyclic ring.

15. The compound of claim 14, wherein each X is phenyl, furan, tetrahydrofuran, 1,3-dioxolane, 2,3-dihydrofuran, tetrahydropyran, benzofuran, isobenzofuran, 1,3-dihydro-isobenzofuran, oxazole, isoxazole, 4,5-dihydroisoxazole, piperidine, pyrrolidine, pyrrolidin-2-one, pyrrole, pyridine, pyrimidine, octahydro-pyrrolo[3,4-b]pyridine, piperazine, pyrazine, morpholine, thiomorpholine, imidazole, imidazolidine-2,4-dione, benzimidazole, 1,3-dihydrobenzimidazol-2-one, indole, thiazole, benzothiazole, thiadiazole, thiophene, tetrahydro-thiophene 1,1-dioxide, diazepine, triazole, triazine, diazabicyclo[2.2.1]heptane, 2,5-diazabicyclo[2.2.1]heptane, or 2,3,4,4a,9,9a-hexahydro-1H-β-carboline, each of which is optionally substituted.

16. The compound of claim 15, wherein X is pyrazine, pyrimidine, thiophene, furan, imidazole, thiazole, triazine, or oxazole.

17. The compound of claim 16, wherein X is pyrazine.

18. The compound of claim 14, wherein each R is H.

19-29. (canceled)

30. The method of claim 1, wherein said compound is (R)-3-(S)-4-nitro-3-(pyrazin-2-yl)butanoyl)-4-benzyloxazolidin-2-one or (R)-3-((R)-4-nitro-3-(pyrazinyl-2-yl)butanoyl)-4-benzyloxazolidin-2-one.

31. The compound of claim 14, wherein said compound is (R)-3-(S)-4-nitro-3-(pyrazin-2-yl)butanoyl)-4-benzyloxazolidin-2-one or (R)-3-((R)-4-nitro-3-(pyrazinyl-2-yl)butanoyl)-4-benzyloxazolidin-2-one.

32. (canceled)
